(19) United States
(12) Patent Application Publication
(10) Pub. No.: US 2008/0096916 A1
(43) Pub. Date: Apr. 24, 2008

(54) Drugs And Prodrugs Useful The Treatment Of Energy Balance In Ruminants

(75) Inventor: Marcus Eugene Kehrli, Ames, IA (US)

Correspondence Address: PFIZER INC., PATENT DEPARTMENT, MS8260-1611, GROTON, CT, 06340, US

(73) Assignee: PFIZER INC., Ann Arbor, MI (US)

(21) Appl. No.: 11/575,431

(22) PCT Filed: Nov. 9, 2005
(86) PCT No.: PCT/IB05/03472

§ 371 Date: Jun. 8, 2007

Related U.S. Application Data
(60) Provisional application No. 60/629,109, filed on Nov. 18, 2004.

(51) Int. Cl. A61K 31/4725; A61P 3/00; C07D 217/02

(52) U.S. Cl. 514/307; 546/146

(57) ABSTRACT

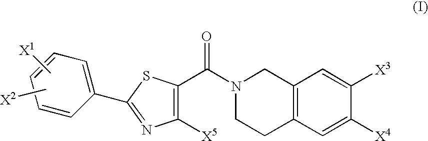

The use of a compound of formula (I) an isomer thereof, a prodrug of said compound or isomer, or a pharmaceutically acceptable salt of said compound, isomer or prodrug, in the manufacture of a medicament for the palliative, prophylactic or curative treatment of negative energy balance in ruminants. The use of a compound of formula 1, in the manufacture of a medicament for the palliative, prophylactic or curative treatment of ruminant disease associated with negative energy balance in ruminants, wherein, preferably, the ruminant disease associated with negative energy balance in ruminants is selected from fatty liver syndrome, dystocia, immune dysfunction, impaired immune function, toxification, primary ketosis, secondary ketosis, downer cow syndrome, indigestion, inappetence, retained placenta, displaced abomasum, mastitis, (endo-)-metritis, infertility, low fertility, and lameness.

### FIELD OF THE INVENTION

The invention described herein relates to the novel use of peroxisome proliferator-activated receptor (PPAR) agonists, in particular PPAR alpha agonists, for the treatment of negative energy balance (NEB) in ruminants, and more particularly for the treatment of disease associated with negative energy balance in ruminants.

### BACKGROUND TO THE INVENTION

The ruminant transition period is defined as the period spanning late gestation to early lactation. This is sometimes defined as from 3 weeks before to three weeks after parturition, but has been expanded to 30 days prepartum to 70 days postpartum (J N Spain and W A Scheer, Tri-State Dairy Nutrition Conference, 2001, 13).

Energy balance is defined as energy intake minus energy output and an animal is described as being in negative energy balance if energy intake is insufficient to meet the demands on maintenance and production (eg milk). A cow in NEB has to find the energy to meet the deficit from its body reserves. Thus cows in NEB tend to lose body condition and liveweight, with cows that are more energy deficient tending to lose condition and weight at a faster rate.

It is important that the mineral and energy balance and overall health of the cow is managed well in the transition period, since this interval is critically important to the subsequent health, production, and profitability in dairy cows.

Ruminants rely almost exclusively on gluconeogenesis in the liver to meet their glucose requirements, since unlike in monogastric mammals, little glucose is absorbed directly from the digestive tract. Feed intake is diminished around calving and insufficient propionate, the major glucogenic precursor formed in the rumen, is available. Catabolism of amino acids from the diet or from skeletal muscle also contributes significantly to glucose synthesis.

Long chain fatty acids (or non esterified fatty acids, NEFAs) are also mobilised from body fat. NEFAs, already elevated from around 7 days prepartum, are a significant source of energy to the cow during the early postpartum period, and the greater the energy deficit the higher the concentration of NEFA in the blood. Some workers suggest that in early lactation (Bell and references therein-see above) mammary uptake of NEFAs accounts for some milk fat synthesis. The circulating NEFAs are taken up by the liver and are oxidised to carbon dioxide or ketone bodies, including 3-hydroxybutyrate, by mitochondria, or reconverted via esterification into triglycerides and stored. In non-ruminant mammals it is thought that entry of NEFAs into the mitochondria is controlled by the enzyme camitine palmitoyltransferase (CPT-1) however, some studies have shown that in ruminants there is little change in activity of CPT-1 during the transition period (Drackley-see above) Furthermore, the capacity of the liver for synthesising very low density lipoproteins to export triglycerides from the liver is limited.

Significantly, if NEFA uptake by the bovine liver becomes excessive, accumulation of ketone bodies can lead to ketosis, and excessive storage of triglycerides may lead to fatty liver. Fatty liver can lead to prolonged recovery for other disorders, increased incidence of health problems, and development of “downer cows” that die.

Thus, fatty liver is a metabolic disease of ruminants, particularly high producing dairy cows, in the transition period that negatively impacts disease resistance (abomasal displacement, lameness), immune function (mastitits, metritis), reproductive performance (oestrus, calving interval, foetal viability, ovarian cysts, metritis, retained placenta), and milk production (peak milk yield, 305 day milk yield). Fatty liver has largely developed by the day after parturition and precedes an induced (secondary) ketosis. It usually results from increased esterification of NEFA absorbed from blood coupled with the low ability of ruminant liver to secrete triglycerides as very low-density lipoproteins.

By improving energy balance, or by treating the negative energy balance, the negative extent of the sequelae will be reduced.

In humans, chronic administration of stimulators of PPAR alpha (peroxisome proliferator activated receptor alpha) activity can provide therapeutic benefits for the treatment of dyslipidemia, coronary artery disease, and certain hereditary enzyme deficiencies (P. T. Ines, P. Gervois, B. Staels, Current Opinion Lipidology, 1999, 10, 2, 151). However, many biological, metabolic and physiological pathways differ between monogastric mammals and ruminants. One typical and important example in the context of this application is the energy metabolism, since microbes in the rumen almost exclusively digest carbohydrates in the food. The main sources for carbohydrates in cows are therefore volatile fatty acids that are re-synthesised to glucose in the liver.

The PPAR alpha gene has also been implicated in a number of metabolic processes by regulating genes involved in gluconeogenesis, ketogenesis, fatty acid uptake and oxidation in mammals, (M. C. Sugden, K. Bulmer, G. F. Gibbons, B. L. Knight, M. J. Holness, Biochem J., 2002, 364, 361).

Most recently Drackley has hypothesised that high fat diets prepartum may have increased PPAR alpha expression, resulting in increased hepatic oxidation and decreased esterification of fatty acids in transition cow liver tissue. However, the interplay of biological processes is complicated as described, and knowledge of the important genes, enzymes and endogenous substrates required to optimise the energy balance in transition cows is limited. Furthermore, it is not known how modification of PPAR expression will effect milk production or quality, lipolysis or gluconeogenesis, since NEFA's are critical substrates for both milk and glucose biosynthesis.

There is a general need for a safe, effective treatment of negative energy balance in ruminants. In particular, there is a need for a treatment for ruminants such as sheep and cattle, more particularly for periparturient sheep and cattle, especially for periparturient dairy cows.

More particularly, there is a need for a safe, effective treatment of ruminant disease associated with negative energy balance in ruminants, which include primary and secondary ketosis, downer cow syndrome, indigestion, inappetence, retained placenta, displaced abomasum, impaired immune function, mastitis, (endo-)-metritis, infertility, low fertility and lameness.

The treatment is preferably administered easily orally or parenterally, preferably does not present residues in meat and/or milk, and preferably does not require a withholding period. It is also preferably non-toxic to feed and animal handlers.

We have discovered a novel use of a compound of formula I, for the palliative, prophylactic or curative treatment of negative energy balance in ruminants. In particular, we have discovered a novel use of a compound of formula I, for the palliative, prophylactic or curative treatment of ruminant disease associated with negative energy balance in ruminants.

One aspect of the invention is the use of a compound of formula I, an isomer thereof, a prodrug of said compound or isomer, or a pharmaceutically acceptable salt of said compound, isomer or prodrug, in the manufacture of a medicament for the palliative, prophylactic or curative treatment of negative energy balance in ruminants.

Another aspect of the invention is a method of palliative, prophylactic or curative treatment of negative energy balance in ruminants, which comprises administration to a ruminant of an effective amount of a compound of formula I, an isomer thereof, a prodrug of said compound or isomer, or a pharmaceutically acceptable salt of said compound, isomer or prodrug.

Further aspects of the invention are as defined in the description and claims.

### DESCRIPTION OF THE DRAWINGS

FIG. 1 shows bovine liver triglyceride content after parturition, and after administration of a PPAR alpha agonist, Compound Z.

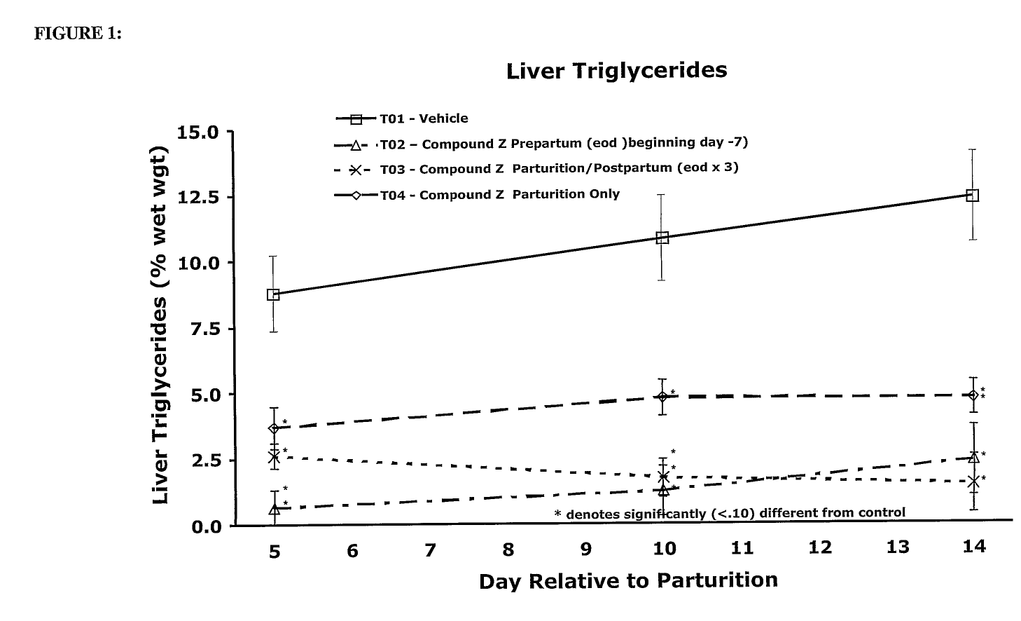

FIG. 2 shows bovine serum NEFA levels after parturition, and after administration of a PPAR alpha agonist, Compound Z.

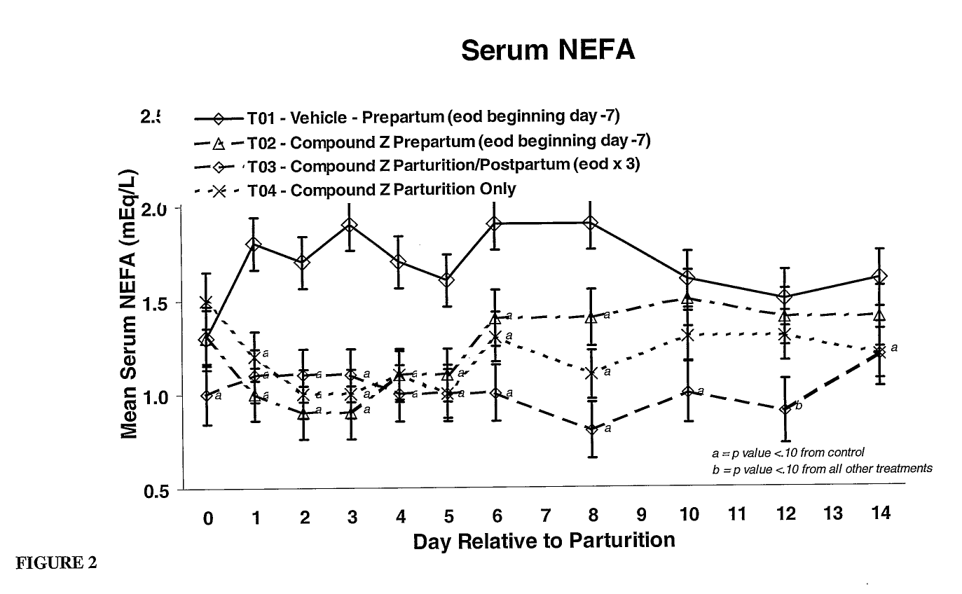

### SUMMARY OF THE INVENTION

The present invention provides the use of a compound of formula I, a prodrug of said compound or a pharmaceutically acceptable salt of said compound or prodrug; in the manufacture of a medicament for the palliative, prophylactic or curative treatment of negative energy balance in ruminants;

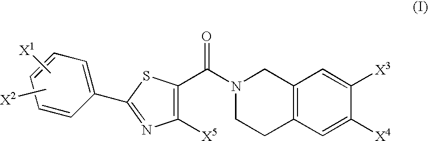

wherein

X¹ and X² are each independently a) hydrogen, b) halo, c) (C₁-C₄)alkyl optionally substituted with one to three fluoro or d) (C₁-C₄)alkoxy optionally substituted with one to three fluoro;

one of X³ and X⁴ is hydrogen and the other is —Y—C(R¹)(R²)—COOH;

Y is —O— or —S—;

R¹ and R² are each independently a) hydrogen or b) (C₁-C₄)alkyl;

X⁵ is —CH₃ or —CF₃.

More particularly, the present invention provides the use of compounds of formula I wherein

X¹ and X² are each independently a) hydrogen, b) —CF₃, c) —OCF₃, d) (C₁-C₄)alkyl, e) —OCH₃ or f) halo;

X³ is —Y—C(R¹)(R²)—COOH and X⁴ is hydrogen;

Y is —O—;

X⁵ is —CH₃.

Even more particularly, the present invention provides the use of such compounds of formula I wherein

one of X¹ and X² is hydrogen and the other is —CF₃;

R¹ and R² are each methyl.

Even more particularly, the present invention provides the use of such compounds of formula I wherein

one of X¹ and X² is hydrogen and the other is —OCF₃;

R¹ and R² are each methyl.

Even more particularly, the present invention provides the use of such compounds of formula I wherein

X¹ and X² are each hydrogen;

R¹ and R² are each methyl.

Even more particularly, the present invention provides the use of such compounds of formula I wherein
one of X¹ and X² is hydrogen and the other is t-butyl;

R¹ and R² are each methyl.

Even more particularly, the present invention provides the use of such compounds of formula I wherein

one of X¹ and X² is hydrogen and the other is —OCH₃;

R¹ and R² are each methyl.

Even more particularly, the present invention provides the use of such compounds of formula I wherein

X¹ and X² are each —CF₃;

R¹ and R² are each methyl.

Even more particularly, the present invention provides the use of such compounds of formula I wherein

X¹ and X² are each —OCH₃;

R¹ and R² are each methyl.

Even more particularly, the present invention provides the use of such compounds of formula I wherein

one of X¹ and X² is hydrogen and the other is halo;

R¹ and R² are each methyl.

In another aspect, the present invention more particularly provides the use of compounds of formula I wherein

X¹ and X² are each independently a) hydrogen, b) —CF₃, c) —OCF₃, d) (C₁-C₄)alkyl, e) —OCH₃ or f) halo;

X³ is hydrogen and X⁴ is —Y—C(R¹)(R²)—COOH;

Y is —O—;

X⁵ is —CH₃.

Even more particularly, the present invention provides the use of such compounds of formula I wherein

one of X¹ and X² is hydrogen and the other is —CF₃;

R¹ and R² are each methyl.

Even more particularly, the present invention provides the use of such compounds of formula I wherein

one of X¹ and X² is hydrogen and the other is —OCF₃;

R¹ and R² are each methyl.

Even more particularly, the present invention provides the use of such compounds of formula I wherein

X¹ and X² are each hydrogen;

R¹ and R² are each methyl.

Even more particularly, the present invention provides the use of such compounds of formula I wherein

one of X¹ and X² is hydrogen and the other is t-butyl;

R¹ and R² are each methyl.

Even more particularly, the present invention provides the use of such compounds of formula I wherein

one of X¹ and X² is hydrogen and the other is —OCH₃;

R¹ and R² are each methyl.

Even more particularly, the present invention provides the use of such compounds of formula I wherein

X¹ and X² are each —CF₃;

R¹ and R² are each methyl.

Even more particularly, the present invention provides the use of such compounds of formula I wherein

X¹ and X² are each —OCH₃;

R¹ and R² are each methyl.

Even more particularly, the present invention provides the use of such compounds of formula I wherein

one of X¹ and X² is hydrogen and the other is halo;

R¹ and R² are each methyl.

Another aspect of the invention is the use of a compound of formula I, in the manufacture of a medicament for the palliative, prophylactic or curative treatment of ruminant disease associated with negative energy balance in ruminants.

Another aspect of the invention is the use of a compound of formula I, in the manufacture of a medicament for the palliative, prophylactic or curative treatment of negative energy balance in ruminants, wherein the excessive accumulation of triglycerides in liver tissue is prevented or alleviated, and/or the excessive elevation of non-esterified fatty acid levels in serum is prevented or alleviated.

Another aspect of the invention is the use of a compound of formula I, in the manufacture of a medicament for the palliative, prophylactic or curative treatment of ruminant disease associated with negative energy balance in ruminants, wherein the excessive accumulation of triglycerides in liver tissue is prevented or alleviated and/or the excessive elevation of non-esterified fatty acid levels in serum is prevented or alleviated.

Preferably, the ruminant disease associated with negative energy balance in ruminants, as mentioned in the aspects of the invention herein, includes one or more diseases selected independently from fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and lameness.

Even more preferably, the ruminant disease associated with negative energy balance in ruminants, as mentioned in the aspects of the invention herein, includes one or more diseases selected from fatty liver syndrome, primary ketosis, downer cow syndrome, (endo-)-metritis and low fertility.

Another aspect of the invention is the use of a compound of formula I, in the improvement of fertility, including decreased return to service rates, normal oestrus cycling, improved conception rates, and improved foetal viability.

Another aspect of the invention is the use of a compound of formula I, in the manufacture of a medicament for the management of effective homeorhesis to accommodate parturition and lactogenesis.

Another aspect of the invention is the use of a compound of formula I, in the manufacture of a medicament for improving or maintaining the functioning of the ruminant liver and homeostatic signals during the transition period.

In one aspect of the invention, the compound of formula I is administered during the period from 30 days prepartum to 70 days postpartum.

In another aspect of the invention, the compound of formula I is administered prepartum and, optionally, also at parturition.

In yet another aspect of the invention, the compound of formula I is administered postpartum.

In yet another aspect of the invention, the compound of formula I is administered at parturition.

More preferably, the compound of formula I is administered during the period from 3 weeks prepartum to 3 weeks postpartum.

In another aspect of the invention, the compound of formula I is administered up to three times during the first seven days postpartum.

Preferably, the compound of formula I is administered once during the first 24 hours postpartum.

In another aspect of the invention, the compound of formula I is administered prepartum and up to four times postpartum.

In another aspect of the invention, the compound of formula I is administered at parturition and then up to four times postpartum.

Another aspect of the invention is the use of the compound of formula I in the manufacture of a medicament for the palliative, prophylactic or curative treatment of negative energy balance in ruminants, and to increase ruminant milk quality and/or milk yield.

In a preferred aspect of the invention, the milk quality increase is seen in a reduction in the levels of ketone bodies in ruminant milk.

In another aspect of the invention, peak milk yield is increased.

Preferably, the ruminant is a cow or sheep.

In another aspect of the invention, an overall increase in ruminant milk yield is obtained during the 305 days of the bovine lactation period.

In another aspect of the invention, an overall increase in ruminant milk yield is obtained during the first 60 days of the bovine lactation period.

Preferably, the overall increase in ruminant milk yield, or the increase in peak milk yield, or the increase in milk quality, is obtained from a dairy cow.

In another aspect of the invention, the increase in ruminant milk quality and/or milk yield is obtained after administration of a compound of formula I to a healthy ruminant.

In another aspect of the invention, there is provided a compound of formula I, for use in veterinary medicine.

In a preferred aspect of the invention, there is provided a compound of formula I, for use in the palliative, prophylactic or curative treatment of negative energy balance in ruminants.

In an even more preferred aspect of the invention, there is provided a compound of formula I, for use in the palliative, prophylactic or curative treatment of ruminant disease associated with negative energy balance in ruminants, wherein, preferably, the disease is selected from fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and lameness.

In another aspect of the invention, there is provided a compound of formula I for use in the palliative, prophylactic or curative treatment of negative energy balance in ruminants, and for increasing ruminant milk quantity and/or quality.

In another aspect of the invention, there is provided a kit for the curative, prophylactic or palliative treatment of negative energy balance in ruminants, comprising:

a) a compound of formula I, and

b) optionally, one or more pharmaceutically acceptable carriers, excipients or diluents, and

c) packaging for containing a) and optionally b)

Preferably, the kit is for the palliative, prophylactic or curative treatment of ruminant diseases associated with negative energy balance in ruminants.

More preferably, the kit is for the palliative, prophylactic or curative treatment of fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and lameness.

Even more preferably, the kit further comprises instructions for the curative, prophylactic or palliative treatment of the negative energy balance or ruminant diseases associated with negative energy balance in ruminants.

The “transition period” means from 30 days prepartum to 70 days postpartum

The term “treating”, “treat”, “treats” or “treatment” as used herein includes prophylactic, palliative and curative treatment.

“Negative energy balance” as used herein means that energy via food does not meet the requirements of maintenance and production (milk).

The term “cow” as used herein includes heifer, primiparous and multiparous cow.

“Healthy ruminant” means where the ruminant does not show signs of the following indications: fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and/or lameness.

Milk “quality” as used herein refers to the levels in milk of protein, fat, lactose, somatic cells, and ketone bodies. An increase in milk quality is obtained on an increase in fat, protein or lactose content, or a decrease in somatic cell levels or ketone bodies levels.

An increase in milk yield can mean an increase in milk solids or milk fat or milk protein content, as well as, or instead of, an increase in the volume of milk produced.

“Excessive accumulation of triglycerides” as used herein means greater than the physiological triglyceride content of 10% w/w in liver tissue.

“Excessive elevation of non-esterified fatty acid levels in serum” as used herein means non-esterified fatty acid levels of greater than 8000 mol/L in serum.

Unless otherwise specified, “prepartum” means 3 weeks before calving until the day of calving.

Unless otherwise specified, “postpartum” means from when the newborn is “expelled” from the uterus to 6 weeks after the newborn was expelled from the uterus.

“At parturition” means the 24 hours after the newborn was expelled from the uterus.

“Periparturient” means the period from the beginning of the prepartum period, to the end of the postpartum period.

By “pharmaceutically acceptable” is meant the carrier, diluent, vehicle, excipient, and/or salt must be compatible with the other ingredients of the formulation, and not deleterious to the recipient thereof.

As used herein, “therapeutically effective amount of a compound” means an amount that is effective to exhibit therapeutic or biological activity at the site(s) of activity in a ruminant, without undue adverse side effects (such as undue toxicity, irritation or allergic response), commensurate with a reasonable benefit/risk ratio when used in the manner of the present invention.

The mention of use of compounds in the present invention, shall at all times be understood to include all active forms of such compounds, including, for example, the free form thereof, e.g., the free acid or base form, and also, all prodrugs, polymorphs, hydrates, solvates, tautomers, stereoisomers, e.g., diastereomers and enantiomers, and the like, and all pharmaceutically acceptable salts as described above, unless specifically stated otherwise. It will also be appreciated that the use of suitable active metabolites of such compounds, in any suitable form, are also included herein.

The expression “prodrug” refers to compounds that are drug precursors which following administration release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the Formula I compounds include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

By halo is meant fluoro, chloro, bromo or iodo.

By alkyl is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl. This term also includes a saturated hydrocarbon (straight chain or branched) wherein a hydrogen atom is removed from each of the terminal carbons.

By alkoxy is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

The carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, C₁-C₃ alkyl refers to alkyl of one to three carbon atoms, inclusive, or methyl, ethyl, propyl and isopropyl, and all isomeric forms and straight and branched forms thereof.

The expression “pharmaceutically-acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

As used herein, the expressions “reaction-inert solvent” and “inert solvent” refers to a solvent or a mixture thereof which does not interact with starting materials, reagents, intermediates or products in a manner which adversely affects the yield of the desired product.

The chemist of ordinary skill will recognize that certain compounds of the present invention will contain one or more atoms, which may be in a particular stereochemical or geometric configuration, giving rise to stereoisomers and configurational isomers. All such isomers and mixtures thereof are included in the present invention. Hydrates and solvates of the compounds of the present invention are also included.

All patents and patent applications referred to herein are hereby incorporated by reference.

### DETAILED DESCRIPTION OF THE INVENTION

In general the compounds useful in the present invention can be made by processes, which include processes analogous to those known in the chemical arts, particularly in light of the description contained herein. Certain processes for the manufacture of the compounds of the present invention are provided as further features of this invention and are illustrated by the following reaction schemes. Other processes are described in the experimental section.

As an initial note, in the preparation of the Formula I compounds, it is noted that some of the preparation methods useful for the preparation of the compounds described herein may require protection of remote functionality (e.g., primary amine, secondary amine, carboxyl in Formula I precursors). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991.

For example, in the reaction schemes below, certain Formula I compounds contain primary amines or carboxylic acid functionalities, which may interfere with reactions at other sites of the molecule if left unprotected. Accordingly, such functionalities may be protected by an appropriate protecting group, which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

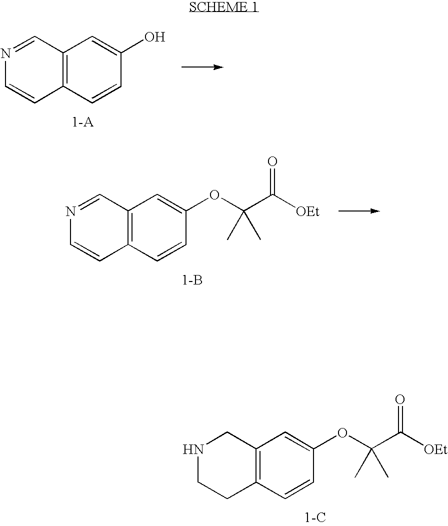

7-Hydroxyisoquinoline of formula 1-A, which is commercially available, ethyl 2-bromoisobutyrate and a base, such as potassium carbonate, are mixed in an appropriate solvent, such as DMF. The reaction mixture is heated to a temperature of about 80° C. to about 120° C., preferably about 95° C., under a nitrogen atmosphere for a period of about 14 hours to about 24 hours, preferably about 18 hours, to give the compound of formula 1-B.

The compound of formula 1-B is reduced, using procedures known in the art, to give the compound I-C. Generally, the compound of formula 1-B is reduced by hydrogenation, preferably at about 50 psi pressure, over a catalyst such as platinum (IV) oxide or Pt/C in an acidic medium such as acetic acid or an acid (such as HCl or H₂SO₄) in an alcoholic solvent at a temperature of about 20° C. to about 30° C., preferably about room temperature, for a period of about 14 to about 24 hours, preferably about 18 hours, to give the compound of formula 1-C.

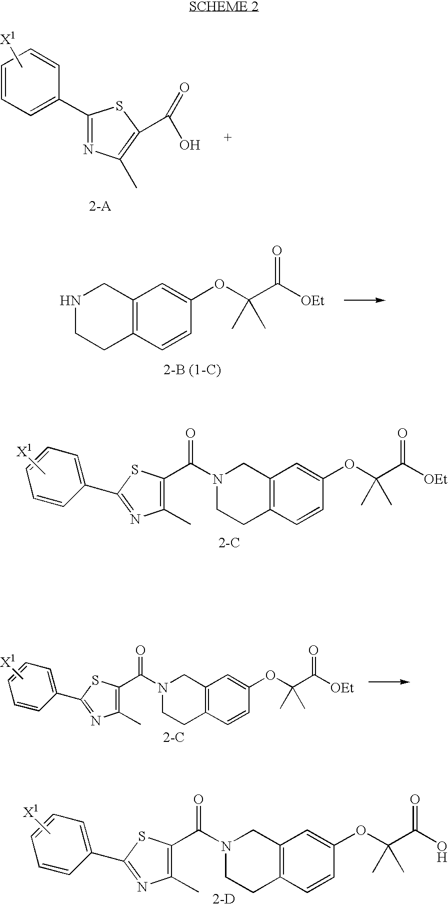

Oxalyl chloride and a solvent, such as DMF, are added to commercially available 4-methyl-2-(substituted-phenyl)-5-thiazolecarboxylic acid of formula 2-A, wherein X¹ is as defined above, in an appropriate solvent, such as methylene chloride, at a temperature of about −5° C. to about 5° C., preferably about 0° C., under a nitrogen atmosphere. The reaction mixture is allowed to warm to room temperature and stirred under a nitrogen atmosphere for a period of about 3 hours to about 6 hours, preferably about 3 hours. The resulting acid chloride is added to the compound of formula 2-B (prepared as the compound of formula 1-C in Scheme 1) in a solvent, such as methylene chloride, and a base, such as triethylamine, at a temperature of about −5° C. to about 5° C., preferably about 0° C., under a nitrogen atmosphere. The reaction mixture is allowed to warm to a temperature of about 20° C. to about 30° C., preferably about room temperature, and stirred under a nitrogen atmosphere for a period of about 14 hours to about 24 hours, preferably about 18 hours, to give the compound of formula 2-C, wherein X¹ is as defined above.

The compound of formula 2-C is hydrolyzed to give the compound of formula 2-D. Alternatively, the hydrolysis may be omitted when the ester is a suitable prodrug for the carboxylic acid. Generally, the ester moiety is hydrolyzed in an aqueous alcoholic solvent such as methanol or ethanol and water with a base, such as potassium carbonate, at a temperature of about 80° C. to about 125° C., preferably about 100° C., for a period of about one to four hours, preferably about 90 minutes, to give the corresponding compound of formula 2-D, wherein X¹ is as defined above.

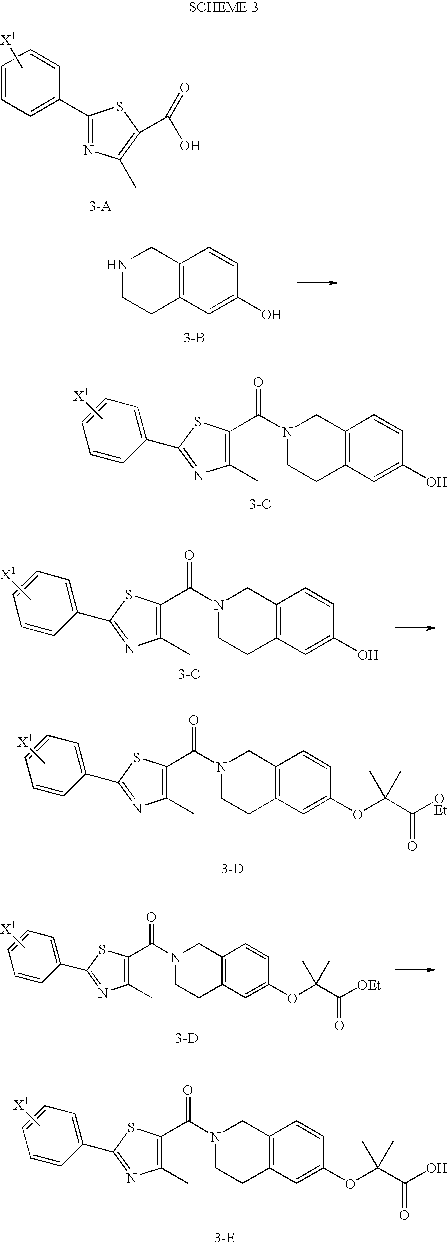

### Scheme 3

Commercially available 4-methyl-2-[substituted-phenyl]-5-thiazolecarboxylic acid of formula 3-A where X¹ is as defined above, 1-hydroxybenzotriazole and 1-(3-dimethylaminopropyl)-3-ethylcarbodiimide hydrochloride are added to 6-hydroxy-1,2,3,4-tetrahydroisoquinoline hydrobromide of formula 3-B (prepared as described in D. J. Sall and G. L. Grunewald, J. Med. Chem., 1987, 30, 2208) in an appropriate base, such as triethylamine, and a solvent, such as methylene chloride. The reaction mixture is stirred under nitrogen atmosphere at a temperature of about 20° C. to about 30° C., preferably about room temperature, for a period of about 14 to about 24 hours, preferably about 18 hours. The reaction is diluted with a solvent, such as methylene chloride, and made acidic with, e.g., citric acid, to give the compound of formula 3-C, wherein X¹ is as defined above.

The compound of formula 3-C is alkylated, using procedures known in the art, to give the compound of formula 3-D, wherein X¹ is as defined above. Generally, the compound of formula 3-C, ethyl 2-bromoisobutyrate and a base, such as potassium carbonate, are mixed in an appropriate solvent, such as DMF. The reaction is heated to a temperature of about 80° C. to about 120° C., preferably about 95° C., under a nitrogen atmosphere for a period of about 14 hours to about 24 hours, preferably about 18 hours. The reaction is concentrated under reduced pressure and an appropriate acid, such as hydrochloric acid, is added to give the compound of formula 3-D, wherein X¹ is as defined above.

The compound of formula 3-D is hydrolyzed to give the corresponding compound of formula 3-E wherein X¹ is as defined above. Alternatively, the hydrolysis may be omitted when the ester is a suitable prodrug for the carboxylic acid. Generally, lithium hydroxide monohydrate in water is added to the compound of formula 3-D in an appropriate solvent, such as THF. The reaction mixture is stirred at a temperature of about 20° C. to about 30° C., preferably about room temperature, for a period of about 14 to about 24 hours, preferably about 18 hours. The reaction is made acidic with an appropriate acid, such as hydrochloric acid, and the solvent, such as THF, is removed under reduced pressure to give the compound of formula 3-E, wherein X¹ is as defined above.

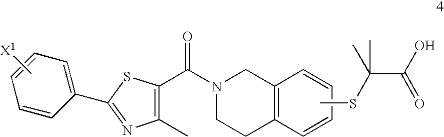

The thiol analogs of formula 4 wherein X¹ is as defined above can be prepared from the corresponding 7-mercapto-1,2,3,4-tetrahydroisoquinoline and 6-mercapto-1,2,3,4-tetrahydroisoquinoline by following the procedures of Schemes 1, 2 and 3 above. 7-mercapto-1,2,3,4-tetrahydroisoquinoline can be prepared as described in U.S. Pat. No. 4,228,170, which is hereby incorporated by reference herein. 6-mercapto-1,2,3,4-tetrahydroisoquinoline can be prepared by starting with commercially available 6-amino-1,2,3,4-tetrahydroisoquinoline and following the procedures described in U.S. Pat. No. 4,228,170.

Preparation of Compounds of Formula I, with Other Permutations of the variables as described above, can be conducted using procedures similar to those described in the schemes above. Additional methods to prepare Formula I compounds would be readily known to one of ordinary skill in the art of organic chemistry and may be further exemplified in the literature and in the Preparations and Examples below.

The starting materials and reagents for the above described reaction schemes are readily available or can be easily synthesized by those skilled in the art using conventional methods of organic synthesis. Some of the preparation methods described herein will require protection of remote functionality (i.e., carboxyl). The need for these protecting groups will vary depending on the nature of the remote functionality and the conditions of the preparation methods and can be readily determined by one skilled in the art. For a general description of protecting groups (e.g., halo(C₁-C₄)alkyl, (C₁-C₄)alkoxymethyl, arylmethyl and tri(C₁-C₄)alkylsilyl) and their use, see T. W. Greene, Protective Groups in Organic Synthesis, John Wiley & Sons, New York, 1991.

Prodrugs of the compounds of Formula I can be prepared according to methods analogous to those known to those skilled in the art. Exemplary processes are described below.

Prodrugs of this invention where a carboxyl group in a carboxylic acid of Formula I is replaced by an ester can be prepared by combining the carboxylic acid with the appropriate alkyl halide in the presence of a base such as potassium carbonate in an inert solvent such as dimethylformamide at a temperature of about 0° C. to about 100° C. for about 1 to about 24 hours. Alternatively, the acid is combined with appropriate alcohol as solvent in the presence of a catalytic amount of acid such as concentrated sulfuric acid at a temperature of about 20° C. to about 100° C., preferably at a reflux, for about 1 hour to about 24 hours. Another method is the reaction of the acid with a stoichiometric amount of the alcohol in the presence of a catalytic amount of acid in an inert solvent such as toluene or tetrahydrofuran, with concomitant removal of the water being produced by physical (e.g., Dean-Stark trap) or chemical (e.g., molecular sieves) means.

Prodrugs of this invention where an alcohol function has been derivatized as an ether can be prepared by combining the alcohol with the appropriate alkyl bromide or iodide in the presence of a base such as potassium carbonate in an inert solvent such as dimethylformamide at a temperature of about 0° C. to about 100° C. for about 1 to about 24 hours. Alkanoylaminomethyl ethers may be obtained by reaction of the alcohol with a bis-(alkanoylamino)methane in the presence of a catalytic amount of acid in an inert solvent such as tetrahydrofuran, according to a method described in U.S. Pat. No. 4,997,984. Alternatively, these compounds may be prepared by the methods described by Hoffman et al. in J. Org. Chem. 1994, 59, 3530.

Glycosides are prepared by reaction of the alcohol and a carbohydrate in an inert solvent such as toluene in the presence of acid. Typically the water formed in the reaction is removed as it is being formed as described above. An alternate procedure is the reaction of the alcohol with a suitably protected glycosyl halide in the presence of base followed by deprotection.

N-(1-hydroxyalkyl) amides and N-(1-hydroxy-1-(alkoxycarbonyl)methyl) amides can be prepared by the reaction of the parent amide with the appropriate aldehyde under neutral or basic conditions (e.g., sodium ethoxide in ethanol) at temperatures between 25° C. and 70° C. N-alkoxymethyl or N-1-(alkoxy)alkyl derivatives can be obtained by reaction of the N-unsubstituted compound with the necessary alkyl halide in the presence of a base in an inert solvent.

The starting materials and reagents for the above described Formula I compounds of the present invention and combination agents, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the Formula I compounds useful in the present invention or intermediates in their synthesis have asymmetric carbon atoms and therefore are enantiomers or diastereomers. Diasteromeric mixtures can be separated into their individual diastereomers on the basis of their physical chemical differences by methods known per se., for example, by chromatography and/or fractional crystallization. Enantiomers can be separated by, for example, chiral HPLC methods or converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Also, an enantiomeric mixture of the Formula I compounds or an intermediate in their synthesis which contain an acidic or basic moiety may be separated into their compounding pure enantiomers by forming a diastereomeric salt with an optically pure chiral base or acid (e.g., 1-phenyl-ethyl amine or tartaric acid) and separating the diasteromers by fractional crystallization followed by neutralization to break the salt, thus providing the corresponding pure enantiomers. All such isomers, including diastereomers, enantiomers and mixtures thereof are considered as part of the present invention. Also, some of the compounds of the present invention are atropisomers (e.g., substituted biaryls) and are considered as part of the present invention.

More specifically, the Formula I compounds useful in the present invention can be obtained by fractional crystallization of the basic intermediate with an optically pure chiral acid to form a diastereomeric salt. Neutralization techniques are used to remove the salt and provide the enantiomerically pure compounds. Alternatively, the Formula I compounds of the present invention may be obtained in enantiomerically enriched form by resolving the racemate of the final compound or an intermediate in its synthesis (preferably the final compound) employing chromatography (preferably high pressure liquid chromatography [HPLC]) on an asymmetric resin (preferably Chiralcel™ AD or OD (obtained from Chiral Technologies, Exton, Pa.)) with a mobile phase consisting of a hydrocarbon (preferably heptane or hexane) containing between 0 and 50% isopropanol (preferably between 2 and 20%) and between 0 and 5% of an alkyl amine (preferably 0.1% of diethylamine). Concentration of the product containing fractions affords the desired materials.

Some of the Formula I compounds of the present invention are acidic and they form a salt with a pharmaceutically acceptable cation. Some of the Formula I compounds of the present invention are basic and they form a salt with a pharmaceutically acceptable anion. All such salts are within the scope of the present invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

Those skilled in the art will recognize that some of the compounds herein can exist in several tautomeric forms. All such tautomeric forms are considered as part of the present invention. For example all enol-keto forms of the compounds of Formula I of the present invention are included in this invention.

In addition, when the Formula I compounds useful in the present invention form hydrates or solvates they are also within the scope of the present invention.

The formula I compounds useful in the present invention, their prodrugs and the salts of such compounds and prodrugs are all adapted to therapeutic use as agents that activate peroxisome proliferator activator receptor (PPAR) activity in ruminants. Thus, it is believed the compounds of the present invention, by activating the PPAR receptor, stimulate transcription of key genes involved in fatty acid oxidation. By virtue of their activity, these agents also reduce plasma levels of triglycerides and NEFA's and prevent accumulation of triglycerides in the liver in ruminants.

The utility of the formula I compounds useful in the present invention, their prodrugs and the salts of such compounds and prodrugs as agents in the treatment of the above described disease/conditions in ruminants is demonstrated by the activity of the compounds of the present invention in the assays described below.

PPAR FRET Assay

Measurement of coactivator recruitment by a nuclear receptor after receptor-ligand association is a method for evaluating the ability of a ligand to produce a functional response through a nuclear receptor. The PPAR FRET (Fluorescence Resonance Energy Transfer) assay measures the ligand-dependent interaction between nuclear receptor and coactivator. GST/PPAR (α,β, and γ) ligand binding domain (LBD) is labeled with a europium-tagged anti-GST antibody, while an SRC-1 (Sterol Receptor Coactivator-1) synthetic peptide containing an amino terminus long chain biotin molecule is labeled with streptavidin-linked allophycocyanin (APC). Binding of ligand to the PPAR LBD causes a conformational change that allows SRC-1 to bind. Upon SRC-1 binding, the donor FRET molecule (europium) comes in close proximity to the acceptor molecule (APC), resulting in fluorescence energy transfer between donor (337 nm excitation and 620 nm emission) and acceptor (620 nm excitation and 665 nm emission). Increases in the ratio of 665 nm emission to 620 nm emission is a measure of the ability of the ligand-PPAR LBD to recruit SRC-1 synthetic peptide and therefore a measure of the ability of a ligand to produce a functional response through the PPAR receptor.

[1] GST/PPAR LBD Expression. The human PPARαLBD (amino acids 235-507) is fused to the carboxy terminus of glutathione S-transferase (GST) in pGEX-6P-1 (Pharmacia, Piscataway, N.J.). The GST/PPARu. LBD fusion protein is expressed in BL21-[DE3]pLysS cells using a 50 uM IPTG induction at room temperature for 16 hr (cells induced at an A₆₀₀ of ˜0.6). Fusion protein is purified on glutathione sepharose 4B beads, eluted in 10 mM reduced glutathione, and dialyzed against 1×PBS at 4° C. Fusion protein is quantitated by Bradford assay (M. M. Bradford, Analst. Biochem. 72:248-254; 1976), and stored at −20° C. in 1×PBS containing 40% glycerol and 5 mM DTT.

[2] FRET Assay. The FRET assay reaction mix consists of 1×FRET buffer (50 mM Tris-Cl pH 8.0, 50 mM KCl, 0.1 mg/ml BSA, 1 mM ethylenediamine tetraacetic acid (EDTA), and 2 mM DTT) containing 20 nM GST/PPARαLBD, 40 nM of SRC-1 peptide (amino acids 676-700, 5′-long chain biotin-CPSSHSSLTERHKILHRLLQEGSPS-NH₂, purchased from American Peptide Co., Sunnyvale, Calif.), 2 nM of europium-conjugated anti-GST antibody (Wallac, Gaithersburg, Md.), 40 nM of streptavidin-conjugated APC (Wallac), and control and test compounds. The final volume is brought to 100 ul with water and transferred to a black 96-well plate (Microfuor B, Dynex (Chantilly, Va.)). The reaction mixes are incubated for 1 hr at 4° C. and fluorescence is read in Victor 2 plate reader (Wallac). Data is presented as a ratio of the emission at 665 nm to the emission at 615 nm.

Selectivity Measurements

Transient transfections assay using the HepG2 hepatoma cell line.

HepG2 cells were transiently transfected with an expression plasmids encoding hPPARα, hPPARβ or mPPARγ chimeric receptors and a reporter containing the yeast upstream activating sequence (UAS) upstream of the viral E1B promoter controlling a luciferase reporter gene. In addition, the plasmid pRSVβ-gal was used to control for transfection efficiency. HepG2 cells were grown in DMEM supplemented with 10% FBS and 1 μM non-essential amino acid. On the first day, cells were split into 100 mm dishes at 2.5×10⁶/dish and incubated overnight at 37 C.°/5% CO₂. On the second day the cells were transiently transfected with plasmid DNA encoding a chimeric receptor, the luciferase reporter gene; and β-gal. For each 100 mm dish, 15 μg of lucifease reporter (PG5E1b) DNA, 15 μg of Gal-4-PPAR chimeric receptor DNA, and 1.5 μg of β-gal plasmid DNA were mixed with 1.4 ml of opti-MEM in the tube. 28 μl of LipoFectamine-2000 reagent was added to 1.4 ml of opti-MEM in the tube, and incubate for 5 min at RT. The diluted Lipofectamine-2000 reagent was combined with the DNA mixture, and incubate for 20 min at RT. After fresh medium was added to each 100 mm dish of cells, 2.8 μl of Lipofectamine-2000-DNA mixture was added dropwise to the 100 mm dish containing 14 ml of medium, and incubate 37° C. overnight. On day three cells were trypsinized off the 100 mm dishes and re-plated on 96 well plates. Cells were plated at 2.5×10⁴ cells per well in 150 μl of media and 50 μL of compound diluted by media was added. The concentrations of reference agents and test compound added were in the range from 50 μM to 50 μM. After addition of compounds, the plates were incubated at 37 C.° for 24 hours. Subsequently cells were washed once with 100 μl of PBS, lysed, and processed for measuring luciferase and β-gal activity using Dual-Light luciferase kit from Tropix®, according to the manufacturer's recommendations, on an EG&G Bethold MicroLumat LB96P luminometer. Hep G2-hBeta EC₅₀ values (“EC₅₀β”) and Hep G2-hAlpha EC₅₀ values, (“EC₅₀α”) were obtained using the GraphPad Prism™ program. EC₅₀ is the concentration at which the PPAR mediated transcriptional response reaches one-half of its maximal response.

Negative Energy Balance

To determine negative energy balance, serum concentrations of NEFAs or ketone bodies, or levels of triglycerides in liver tissues, are measured. Higher than ‘normal’ levels of NEFA's and/or triglycerides and/or ketone bodies are indicators of negative energy balance. Levels considered ‘higher than normal’ or ‘excessive’ are:

NEFA's>800 □mol/L in serum.

Triglycerides>10% w/w in liver tissue.

Ketone bodies>1.2 □mol/L in serum.

Determination of Changes in Blood Non-Esterified Fatty Acid (NEFA) Concentrations and Liver Triglycerides Levels:

Compounds are administered once or several times in the transition period at dose levels predicted to be effective by comparing results of in-vitro receptor affinity tests in laboratory species and pharmacokinetic evaluations in cattle. NEFA levels are determined via standard laboratory methods, for example, using the commercial WAKO NEFA kit (Wako Chemical Co., USA, Dallas, Tex., 994-75409), and liver triglyceride content is determined using the method as described in the literature (J. K. Drackley, J. J. Veenhuizen, M. J. Richard and J. W. Young, J Dairy Sci, 1991, 74, 4254)).

All animals may be obtained from a commercial dairy farm approximately thirty days prior to anticipated calving date. The cows are moved into separate building, approximately 10-14 days prior to their anticipated calving dates and switched to the TMR-Close-Up dry diet. Enrolment of animals in the study begins approximately 7 days prior to their anticipated calving dates. The animals may be moved to the “on-test” pen, weighed and are locked each AM into feed stanchions. At that time, appropriate doses are administered and appropriate blood samples obtained (see table below).

| | | | Pre Partum Dosing | | |
|---|---|---|---|---|---|
| | | | (every other day = | | |
| | | Animals per | eod − beginning | Treatment | Post Partum Dosing |
| Treatment | Dosage | Treatment | targeted day − 7) | at Calving | (eod 4 doses) |
| T01 | — | 9 | X | X | |
| Vehicle Control | | | | | |
| T02 | 0.5 mg/kg | 8 | X | X | |
| Compound Z | | | | | |
| T03 | 0.5 mg/kg | 11 | | X | X |
| Compound Z | | | | | |
| T04 | 0.5 mg/kg | 9 | | X | |
| Compound Z | | | | | |

As soon as possible post-calving (˜30 minutes) the cow is transferred to the freestall barn for the next scheduled milking (6:00 hrs and 19:00 hrs). Treatments on postpartum animals are administered every other day through day 8. Pre and post-calving NEFA samples are analyzed using the WAKO NEFA-C test kit (#994-75409). Post-calving liver biopsies are performed on all cows on days 5, 10 and 14 post-calving. Tissues are transported on ice and stored frozen at −70° F. At the conclusion of the study, samples are analysed of liver triglyceride levels using the method described by Drackley, J. K. et al. (1991, J Dairy Sci (74):4254-4264).

In a related experiment to illustrate the class effect, all animals treated with Compound Z, a PPAR alpha agonist, exhibited significantly lower serum NEFA levels from Day 1 (after calving) until Day 6 of the study as compared to controls. In addition, animals in treatment group T03 exhibited significantly lower serum NEFA levels compared to controls at all timepoints. All treatment regimens significantly lowered liver triglyceride levels compared to placebo at all time points measured (Days 5, 10 and 14 postcalving).

Ketone Bodies

Levels of ketone bodies in serum can be measured by standard methods well known to the person skilled in the art, for example, by using the commercially available kits for this purpose, including Sigma BHBA kit of order number 310-A.

Milk Content:

Machines to assay for milk protein, fat, or lactose content are commercially available (MilkoScan™ 50, MilkoScan™ 4000, MilkoScan™ FT 6000 available from Foss Group).

Machines to assay for somatic cell content are also commercially available (Fossomatic™ FC, Fossomatic™ Minor available from Foss Group).

Compounds used in this invention may be administered alone or in combination with one or more other compounds of the invention or in combination with one or more other drugs (or as any combination thereof).

For example, compounds of this invention can also be mixed with one or more biologically active compounds or agents selected from sedatives, analgesics, antiinflammatories, analeptics, antibacterials, antidiarrhoeals, anti-endotoxin, antifungals, respiratory stimulants, corticosteroids, diuretics, parasiticides, electrolyte preparations and nutritional supplements, growth promoters, hormones, and metabolic disease treatments, giving an even broader spectrum of veterinary or agricultural utility.

Examples of suitable active compounds or agents are found below:

Amylase inhibitors: Acarbose;

Sedative: xylazine,

Analgesics and antiinflammatories: Lignocaine, Procaine, flunixin, oxytetracycline, ketoprofen, meloxicam and carprofen.

Analeptics: Etamiphylline, Doxapram, Diprenorphine, Hyoscine, Ketoprofen, Meloxicam, Pethidine, Xylazine and Butorphanol,

Antibacterials: Chlortetracycline, Tylosin, Amoxycillin, Ampicillin, Aproamycin, Cefquinome, Cephalexin, Clavulanic acid, Plorfenicol, Danofloxacin, Enrofloxacin, Marbofloxacin, Framycetin, Procaine penicillin, procaine benzylpenicillin, Benzathine penicillin, sulfadoxine, Trimethoprim, sulphadimidine, baquiloprim, streptomycin, dihydrostreptomycin, sulphamethoxypyridazine, sulphamethoxypuridazine, oxytetracycline, flunixin, tilmicosin, cloxacillin, ethyromycin, neomycin, nafcillin, Aureomycin, lineomycin, cefoperazone, cephalonium, oxytetracycline, formosulphathiazole, sulphadiazine and zinc.

Antidiarrhoeals: Hyoscine, Dipyrone, charcoal, attapulgite, kaolin, Isphaghula husk,

Anti-endotoxins: Flunixin, ketoprofen,

Antifungals: Enilconazole, Natamycin,

Respiratory stimulants: florfenicol,

Corticosteroids: dexamethasone, betamethasone,

Diuretics: frusemide,

Parasiticides—amitraz, deltamethrin, moxidectin, doramectin, alpha cypermethrin, fenvalerate, eprinomectin, permethrin, ivermectin, abamectin, ricobendazole, levamisole, febantel, triclabendazole, fenbendazole, albendazole, netobimin, oxfenazole, oxyclozanide, nitroxynil, morantel, Electrolyte preparations and nutritional supplements: dextrose, lactose, propylene glycol, whey, glucose, glycine, calcium, cobalt, copper, iodine, iron, magnesium, manganese, phosphorous, selenium, zinc, Biotin, vitamin B12, Vitamin E, and other vitamins,

Growth Promoters: monensin, flavophospholipol, bambermycin, salinomycin, tylosin,

Hormones: chorionic gonadotrophin, serum gonadotrophin, atropine, melatonin, oxytocin, dinoprost, cloprostenol, etiproston, luprostiol, buserelin, oestradiol, progesterone, and bovine somatotropin, and

Metabolic Disease Treatments: calcium gluconate, calcium borogluconate, propylene glycol, magnesium sulphate.

Compounds of this invention can also be mixed with one or more biologically active compounds or agents selected from antiprotozoals such as imidocarb, bloat remedies such as dimethicone and poloxalene, and probiotics such as Lactobacilli and streptococcus.

Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

With respect to their use in ruminants, the compounds may be administered alone or in a formulation appropriate to the specific use envisaged.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the use invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001).

The compounds of the invention may also be administered directly into the blood stream, into muscle, or into an internal organ. Suitable means for parenteral administration include bolus, intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

The compounds of the invention may also be administered topically to the skin or mucosa, that is, dermally or transdermally. Typical formulations for this purpose include drenches, gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999). Pour-on or spot-on formulations may be prepared by dissolving the active ingredient in an acceptable liquid carrier vehicle such as butyl digol, liquid paraffin or a non-volatile ester, optionally with the addition of a volatile component such as propan-2-ol. Alternatively, pour-on, spot-on or spray formulations can be prepared by encapsulation, to leave a residue of active agent on the surface of the animal. Injectable formulations may be prepared in the form of a sterile solution which may contain other substances, for example enough salts or glucose to make the solution isotonic with blood.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or BHMC), blisters and cartridges for use in an inhaler or insufflator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 to 1000 μg of the compound of formula (I). The overall daily dose will typically be in the range 100 μg to 100 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate. Silicone rubber based intravaginal devices can be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysacchalide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Acceptable liquid carriers include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending the active ingredient in the liquid carrier such that the final formulation contains from 0.01 to 10% by weight of the active ingredient.

Such formulations are prepared in a conventional manner in accordance with standard veterinary practice.

These formulations will vary with regard to the weight of active compound contained therein, depending on the species of host animal to be treated, the severity and type of infection and the body weight of the host. For parenteral, topical and oral administration, typical dose ranges of the active ingredient are 0.05 to 5 mg per kg of body weight of the animal. Preferably the range is 0.01 to 1 mg per kg.

As an alternative the compounds may be administered to a ruiminant with the drinking water or feedstuff and for this purpose a concentrated feed additive or premix may be prepared for mixing with the normal animal feed or drink.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to ruminants, the total daily dose of the compounds of the invention is typically in the range 0.05 mg/kg to 5 mg/kg depending, of course, on the mode of administration. For example, oral administration may require a total daily dose of from 0.05 mg/kg to 5 mg/kg, while an intravenous dose may only require from 0.01 mg/kg to 1 mg/kg. The total daily dose may be administered in single or divided doses. The veterinarian will readily be able to determine doses for individual ruminants according to age, weight and need.

### FORMULATION EXAMPLES

In the formulations which follow, “active ingredient” means a compound used in the present invention.

Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

Formulation 2: Tablets—A Tablet Formulation is Prepared Using the Ingredients Below:

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in water) | 4 |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

Formulation 4: Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form a smooth paste. The benzoic acid solution, flavor, and color are diluted with some of the water and added, with stirring. Sufficient water is then added to produce the required volume.

An intravenous formulation is prepared as follows:

Formulation 5: Intravenous Solution

| Ingredient | Quantity | |
|---|---|---|
| Active ingredient dissolved in ethanol 1% | 20 | mg |
| Intralipid ™ emulsion | 1,000 | mL |

The solution of the above ingredients is intravenously administered to a patient at a rate of about 1 mL per minute.

Formulation 6: Soft Gelatin Capsule with Oil Formulation Soft Gelatin Capsules are Prepared Using the Following:

| Ingredient | Quantity (mg/capsule) |
|---|---|
| Active ingredient | 10-500 |
| Olive Oil or Miglyol ™ Oil | 500-1000 |

The active ingredient above may also be a combination of therapeutic agents.

### General Experimental Procedures

NMR spectra were recorded on a Varian XL-300 (Varian Co., Palo Alto, Calif.), a Bruker AM-300 spectrometer (Bruker Co., Billerica, Mass.) or a Varian Unity 400 at ambient temperature. Chemical shifts are expressed in parts per million (8) relative to residual solvent as an internal reference. The peak shapes are denoted as follows: s, singlet; d, doublet; dd, doublet of doublets, t, triplet, q, quartet; m, multiplet; brs=broad singlet; 2s, two singlets. Atmospheric pressure chemical ionization (APCI) mass spectra in alternating positive and negative ion mode were obtained on a Fisons Platform II Spectrometer, Fisons Instruments Manchester U.K.). Chemical ionization mass spectra were obtained on a Hewlett-Packard 5989 instrument (Hewlett-Packard Co., Palo Alto, Calif.) (ammonia ionization, PBMS). Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and the intensity of only the lower mass ion is given. Optical rotations were determined on a Perkin-Elmer 241 polarimeter (Perkin-Elmer Instruments, Norwalk, Conn.) using the sodium D line (λ=589 nm) at the indicated temperature and are reported as follows [α]_{D}^{temp}, concentration (c=g/100 mL), and solvent.

Column chromatography was performed with either Baker Silica Gel (40 μm) (J. T. Baker, Phillipsburg, N.J.) or Silica Gel 50 (EM Sciences, Gibbstown, N.J.) in glass columns or in Flash 40 (Biotage, Dyar Corp. Charlottesville, Va.) columns under low nitrogen pressure. Radial Chromatography was performed using a Chromatron (model 7924T, Harrison Research, Palo Alto, Calif.). Unless otherwise specified, reagents were used as obtained from commercial sources. Dimethylformamide, 2-propanol, tetrahydrofuran, toluene and dichloromethane used as reaction solvents were the anhydrous grade supplied by Aldrich Chemical Company (Milwaukee, Wis.). Microanalyses were performed by Schwarzkopf Microanalytical Laboratory, Woodside, N.Y. The terms “concentrated” and “evaporated” refer to removal of solvent at 5-200 mm of mercury pressure on a rotary evaporator with a bath temperature of less than 45° C. Reactions conducted at “0-20° C.” or “0-25° C.” were conducted with initial cooling of the vessel in an insulated ice bath which was then allowed to warm to room temperature. The abbreviation “min” and “h” stand for “minutes” and “hours” respectively. The abbreviation “rt” stands for “room temperature.” Other abbreviations, which would be readily understandable to one of ordinary skill in the art, are used, such as the following: “N₂” stands for nitrogen; “CH₂Cl₂” stands for dichloromethane; “THF” stands for tetrahydrofuran; “NaHCO₃” stands for sodium bicarbonate.

### Preparation 1

### 2-(Isoquinolin-7-yloxy)-2-methyl-propionic acid ethyl ester

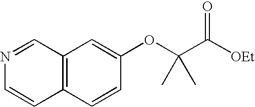

7-Hydroxyisoquinoline (500 mg, 3.4 mmol), ethyl 2-bromoisobutyrate (3 g, 15 mmol) and potassium carbonate (2.1 g, 15 mmol) were mixed in 7 ml of anhydrous DMF. The reaction was heated to 95° C. under N₂ for 18 hrs. The reaction was concentrated under reduced pressure and purified by flash column chromatography (33% EtOAc/Hexanes) to yield 470 mg (53%) of the title product of this preparation as a yellow oil.

¹H NMR (400 MHz, CDCl₃) δ 9.08 (s, 1H), 8.41 (d, 1H), 7.72 (d, 1H), 7.57 (d, 1H), 7.33 (dd, 1H), 7.13 (d, 1H), 4.25 (q, 2H), 1.69 (s, 6H), 1.22 (t, 3H).

### Preparation 2

### 2-(1,2,3,4,4a,8a-Hexahydro-isoquinolin-7-yloxy)-2-methyl-propionic acid ethyl ester

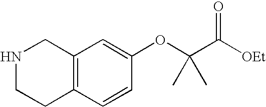

The title product of Preparation 1 (470 mg, 1.8 mmol) and platinum oxide (21 mg, 0.09 mmol) were mixed in 10 ml glacial acetic acid, under 50 psi of H₂ at room temperature for 18 hrs. The reaction was filtered though Celite and concentrated under reduced pressure. The residue was diluted with EtOAc and made basic with 1 N NaOH. The organic layer was separated and the aqueous phase was extracted with 3× EtOAc. The organic phases were combined, washed with brine, dried over Na₂SO₄, filtered and concentrated to yield 443 mg (93%) of the title product of this preparation as a yellow oil.

¹H NMR (400 MHz, CDCl₃) δ 6.94 (d, 1H), 6.63 (dd, 1H), 6.50 (d, 1H), 4.23 (q, 2H), 3.92 (s, 2H), 3.09 (t, 2H), 2.70 (t, 2H), 1.56 (s, 6H), 1.25 (t, 3H).

### Preparation 3

### 2-Methyl-2-{2-[4-methyl-2-(4-trifluoromethyl-phenyl)-thiazole-5-carbonyl]-1,2,3,4,4a,8a-hexahydro-isoquinolin-7-yloxy}-propionic acid ethyl ester

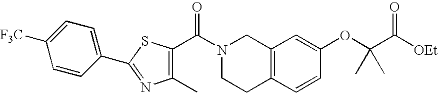

Oxalyl chloride (310 μl, 3.55 mmol) and 5 drops of DMF were added to 4-methyl-2-[4-(trifluoromethyl)phenyl]-5-thiazolecarboxylic acid (1.02 g, 3.55 mmol) (commercially available) in 50 ml of methylene chloride at 0° C. under N₂. The reaction was allowed to warm to room temperature and stirred under N₂ for 3 hrs. The resulting acid chloride was added dropwise to the title product of Preparation 2 (935 mg, 3.55 mmol) in 50 ml methylene chloride and triethylamine (500 μl, 3.55 mmol) at 0° C. under N₂. The reaction was allowed to warm to room temperature and stirred under N₂ for 18 hrs. The reaction was diluted with CH₂Cl₂ and washed with saturated NaHCO₃. The organic phase was washed with brine, dried over Na₂SO₄, filtered and concentrated. The residue was purified by flash column chromatography (33% EtOAc/Hexanes) to yield 1.31 g (70%) of the title product of this preparation.

MS m/z 533 (M+1);

¹H NMR (400 MHz, CDCl₃) δ 8.05 (d, 2H), 7.71 (d, 2H), 7.03 (d, 1H), 6.70 (dd, 1H), 6.62 (bs, 1H), 4.74 (bs, 2H), 4.23 (q, 2H), 3.81 (bs, 2H), 2.88 (s, 2H), 2.52 (s, 3H), 1.57 (s, 6H), 1.25 (t, 3H).

### Example 1

### 2-Methyl-2-{2-[4-methyl-2-(4-trifluoromethyl-phenyl)-thiazole-5-carbonyl]-1,2,3,4-tetrahydro-isoquinolin-7-yloxy}-propionic acid

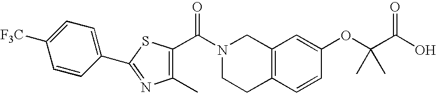

The title product of Preparation 3 (3.12 g, 5.86 mmol) was mixed in 50 ml of 3:1 mixture of EtOH and water. Potassium carbonate (3.24 g, 23.4 mmol) was added and reaction heated to 100° C. for 90 minutes. Reaction was cooled to room temperature and concentrated under reduced pressure. The residue was partitioned between EtOAc and 1N HCl. The organic phase was washed with water, brine, dried over Na₂SO₄, filtered and concentrated. The residue was purified by flash column chromatography (1% NOH/15% MeOH/CH₂Cl₂) to yield 2.27 g (77%) of the title product of this example as a white solid.

MS m/z 505 (M+1);

¹H NMR (400 MHz, CDCl₃) δ 8.04 (d, 2H), 7.70 (d, 2H), 7.08 (d, 1H), 6.81 (dd, 1H), 6.71 (bs, 1H), 4.75 (bs, 2H), 3.84 (bs, 2H), 2.90 (bs, 2H), 2.51 (s, 3H), 1.59 (s, 6H).

Examples 2 to 10 were prepared from analogous starting materials using methods analogous to those described in Example 1:

### Example 2

2-{2-[2-(4-tert-Butyl-phenyl)-4-methyl-thiazole-5-carbonyl]-1,2,3,4-tetrahydro-isoquinolin-7-yloxy}-2-methyl-propionic acid

MS m/z 493 (M+1);

¹H NMR (400 MHz CDCl₃) δ 7.80 (d, 2H), 7.42 (d, 2H), 6.94 (d, 1H), 6.71 (d, 2H), 6.65 (bs, 1H), 4.66 (bs, 2H), 3.73 (bs, 2H), 2.79 (bs, 2H), 2.43 (s, 3H), 1.45 (s, 6H), 1.32 (s, 9H).

### Example 3

### 2-{2-[2-(4-Methoxy-phenyl)-4-methyl-thiazole-5-carbonyl]-1,2,3,4-tetrahydro-isoquinolin-7-yloxy}-2-methyl-propionic acid

MS m/z 467 (M+1);

¹H NMR (400 MHz CDCl₃) δ 7.81 (d, 2H), 6.92 (m, 3H), 6.70 (d 1H), 6.63 (s, 1H), 4.65 (s, 2H), 3.83 (s, 3H), 3.77 (s, 2H), 2.79 (s, 2H), 2.42 (s, 3H), 1.45 (s, 6H).

### Example 4

### 2-{2-[2-(3,5-Bis-trifluoromethyl-phenyl)-4-methyl-thiazole-5-carbonyl]-1,2,3,4-tetrahydro-isoquinolin-7-yloxy}-2-methyl-propionic acid

MS m/z 573 (M+1);

¹H NMR (400 MHz CDCl₃) δ 8.34 (s, 2H), 7.93 (s, 1H), 7.03 (d, 1H), 6.77 (d, 1H), 6.67 (bs, 1H), 4.72 (bs, 2H), 3.79 (bs, 2H), 2.87 (bs, 2H), 2.51 (s, 3H), 1.52 (s, 6H).

### Example 5

### 2-Methyl-2-{2-[4-methyl-2-(3-trifluoromethyl-phenyl)-thiazole-5-carbonyl]-1,2,3,4-tetrahydro-isoquinolin-7-yloxy}-propionic acid

MS m/z 505 (M+1);

¹H NMR (400 MHz CDCl₃) δ 8.18 (s, 1H), 8.04 (d, 1H), 7.69 (d, 1H), 7.56 (t, 1H), 6.99 (d, 1H), 6.74 (dd, 1H), 6.67 (bs, 1H), 4.70 (bs, 2H), 3.76 (bs, 2H), 2.83 (bs, 2H), 2.47 (s, 3H), 1.49 (s, 6H).

### Example 6

### 2-Methyl-2-{2-[4-methyl-2-(2-trifluoromethyl-phenyl)-thiazole-5-carbonyl]-1,2,3,4-tetrahydro-isoquinolin-7-yloxy}-propionic acid

¹H NMR (400 MHz CDCl₃) δ 7.80 (d, 1H), 7.61 (m, 3H), 7.00 (d, 1H), 6.72 (d, 1H), 6.66 (s, 1H), 4.72, (s 2H), 3.77 (s, 2H), 2.86 (s, 2H), 2.49 (s, 3H), 1.47 (s, 6H).

### Example 7

### 2-{2-[2-(4-Chloro-phenyl)-4-methyl-thiazole-5-carbonyl]-1,2,3,4-tetrahydro-isoquinolin-7-yloxy}-2-methyl-propionic acid

MS m/z 471 (M+1);

¹H NMR (400 MHz CDCl₃) δ 7.91 (d, 2H), 7.43 (d, 2H), 7.09 (d, 1H), 6.81 (d, 1H), 6.67 (bs, 1H), 4.75 (bs, 2H), 3.85 (bs, 2H), 2.91 (s, 2H), 2.50 (s, 3H), 1.59 (s, 6H).

### Example 8

### 2-{2-[2-(3,4-Dimethoxy-phenyl)-4-methyl-thiazole-5-carbonyl]-1,2,3,4-tetrahydro-isoquinolin-7-yloxy}-2-methyl-propionic acid

MS m/z 497 (M+1);

¹H NMR (400 MHz CD₃OD) δ 7.58 (s, 1H), 7.51 (d, 1H), 7.09 (d, 1H), 7.05 (d, 1H), 6.75 (m, 2H), 4.75 (bs, 2H), 3.92 (s, 3H), 3.90 (s, 3H), 3.88 (bs, 2H), 2.91 (bs, 2H), 2.42 (s, 3H), 1.55 (s, 6H).

### Example 9

### 2-Methyl-2-{2-[4-methyl-2-(4-trifluoromethoxy-phenyl)-thiazole-5-carbonyl]-1,2,3,4-tetrahydro-isoquinolin-7-yloxy}-propionic acid

MS m/z 521 (M+1);

¹H NMR (400 MHz CDCl₃) δ 7.92 (d, 2H), 7.26 (d, 2H), 6.97 (d, 1H), 6.72 (d, 1H), 6.65 (bs, 1H), 4.68 (bs, 2H), 3.76 (bs, 2H), 2.83 (bs, 2H), 2.45 (s, 3H), 1.46 (s, 6H).

### Example 10

### 2-Methyl-2-[2-(4-methyl-2-phenyl-thiazole-5-carbonyl)-1,2,3,4-tetrahydro-isoquinolin-7-yloxy]-propionic acid

MS m/z 437 (M+1);

¹H NMR (400 MHz CDCl₃) δ 7.87 (m, 2H), 7.42 (m 3H), 6.89 (d, 1H), 6.67 (d, 1H), 6.61 (bs, 1H), 4.65 (bs, 2H), 3.70 (bs, 2H), 2.76 (bs, 2H), 2.41 (s, 3H), 1.34 (s, 6H).

### Preparation 4

### (6-Hydroxy-3,4-dihydro-1H-isoquinolin-2-yl)-[4-methyl-2-(4-trifluoromethyl-phenyl)-thiazol-5-yl]-methanone

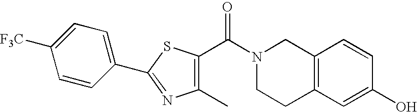

4-Methyl-2-[4-(trifluoromethyl)phenyl]-5-thiazolecarboxylic acid (450 mg, 1.6 mmol), 1-hydroxybenzotriazole (260 mg, 1.9 mmol) and 1-(3-dimethylaminopropyl)-3-ethylcarbodiimide hydrochloride (370 mg, 1.9 mmol) were added to 6-hydroxy-1,2,3,4-tetrahydroisoquinoline hydrobromide (300 mg, 1.3 mmol) in 2 ml of triethylamine and 6 ml of methylene chloride. The reaction was stirred under N₂ at RT for 18 hrs. The reaction was diluted with 100 ml CH₂Cl₂ and made acidic with 10% citric acid. The organic layer was separated and the aqueous phase was extracted with 2×100 ml CH₂Cl₂. The organic phases were combined, washed with H₂O, brine, dried over Na₂SO₄, filtered and concentrated. The residue was purified by flash column chromatography (40% EtOAc/Hexanes) to yield 229 mg (42%) of the title product of this preparation as a white solid.

¹H NMR (400 MHz, CDCl₃) δ 8.05 (d, 2H), 7.71 (d, 2H), 6.94 (m, 1H), 6.70 (d, 1H), 6.65 (d, 1H), 4.72 (bs, 2H), 3.84 (bs, 2H), 2.90 (s, 2H), 2.52 (s, 3H).

### Preparation 5

### 2-Methyl-2-{2-[4-methyl-2-(4-trifluoromethyl-phenyl)-thiazole-5-carbonyl]-1,2,3,4-tetrahydro-isoquinolin-6-yloxy}-propionic acid ethyl ester

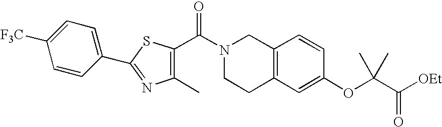

The title product of Preparation 4 (218 mg, 0.52 mmol), ethyl 2-bromoisobutyrate (457 mg, 2.3 mmol) and potassium carbonate (318 mg, 2.3 mmol) were mixed in 2 ml of anhydrous DMF. The reaction was heated to 95° C. under N₂ for 18 hrs. The reaction was concentrated under reduced pressure and 50 ml of 1N HCl was added. The aqueous phase was extracted with EtOAc (3×100 ml). The organic phases were combined, washed with H₂O, brine, dried over Na₂SO₄, filtered and concentrated. The residue was purified by flash column chromatography (25% EtOAc/Hexanes) to yield 194 mg (70%) of the title product of this preparation.

¹H NMR (400 MHz, CDCl₃) δ 8.05 (d, 2H), 7.71 (d, 2H), 6.99 (bs, 1H), 6.70 (d, 1H), 6.67 (s, 1H), 4.72 (bs, 2H), 4.24 (q, 2H), 3.83 (bs, 2H), 2.89 (s, 2H), 2.51 (s, 3H), 1.59 (s, 6H), 1.28 (t, 3H).

### Example 11

### 2-Methyl-2-{2-[4-methyl-2-(4-trifluoromethyl-phenyl)-thiazole-5-carbonyl]-1,2,3,4-tetrahydro-isoquinolin-6-yloxy}-propionic acid

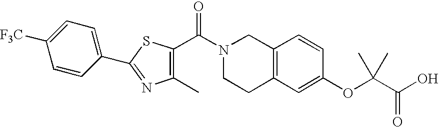

Lithium hydroxide monohydrate (151 mg, 3.6 mmol) in 4 ml of H₂O was added to the title product of Preparation 5 (190 mg, 0.36 mmol) in 4 ml of THF. The reaction was stirred at RT for 18 hrs. The reaction was made acidic with 1N HCl and the THF was removed under reduced pressure. The aqueous phase was extracted with EtOAc (3×50 ml). The organic phases were combined, washed with brine, dried over Na₂SO₄, filtered and concentrated. The residue was purified by flash column chromatography (1% NH₄OH/10% MeOH/CH₂Cl₂) to yield 113 mg (62%) of the title product of this example as a white solid.

MS m/z 505 (M+1);

¹H NMR (400 MHz, CDCl₃) δ 8.04 (d, 2H), 7.71 (d, 2H), 7.01 (bs, 1H), 6.80 (d, 1H), 6.76 (s, 1H), 4.75 (bs, 2H), 3.84 (bs, 2H), 2.91 (m, 2H), 2.51 (s, 3H), 1.60 (s, 6H).

Examples 12 to 14 were prepared from analogous starting materials using methods analogous to those described in Example 11:

### Example 12

### 2-{2-[2-(4-Methoxy-phenyl)-4-methyl-thiazole-5-carbonyl]-1,2,3,4-tetrahydro-isoquinolin-6-yloxy}-2-methyl-propionic acid

MS m/z 467 (M+1);

¹H NMR (400 MHz CDCl₃) δ 7.79 (d, 2H), 6.86 (m, 3H), 6.65 (m, 2H), 4.59 (s, 2H), 3.80 (s, 3H), 3.68 (s, 2H), 2.75 (s, 2H), 2.40 (s, 3H), 1.38 (s, 6H).

### Example 13

### 2-Methyl-2-{2-[4-methyl-2-(3-trifluoromethyl-phenyl)-thiazole-5-carbonyl]-1,2,3,4-tetrahydro-isoquinolin-6-yloxy}-propionic acid

MS m/z 505 (M+1);

¹H NMR (400 MHz CDCl₃) δ 8.17 (s 1H), 8.03 (d, 1H), 7.68 (d, 1H), 7.55 (t, 1H), 6.90 (bs, 1H), 6.72 (m, 2H), 4.67 (bs, 2H), 3.73 (bs, 2H), 2.81 (s, 2H), 2.47 (s, 3H), 1.46 (s, 6H).

### Example 14

### 2-Methyl-2-{2-[4-methyl-2-(2-trifluoromethyl-phenyl)-thiazole-5-carbonyl]-1,2,3,4-tetrahydro-isoquinolin-6-yloxy}-propionic acid

MS m/z 505 (M+1);

¹H NMR (400 MHz CDCl₃) δ 7.79 (d, 1H), 7.61 (m, 3H), 6.97 (bs, 1H), 6.76 (m, 2H), 4.71 (bs, 2H), 3.78 (bs, 2H), 2.81 (s, 2H), 2.50 (s, 3H), 1.51 (s, 6H).

1. The use of a compound of formula I: 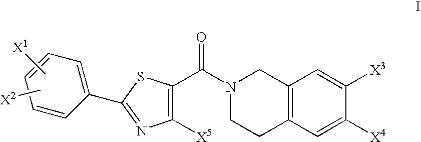
a prodrug of said compound or a pharmaceutically acceptable salt of said compound or prodrug; wherein
X¹ and X² are each independently a) hydrogen, b) halo, c) (C₁-C₄)alkyl optionally substituted with one to three fluoro or d) (C₁-C₄)alkoxy optionally substituted with one to three fluoro;
one of X³ and X⁴ is hydrogen and the other is —Y—C(R¹)(R²)—COOH;
Y is —O— or —S—;
R¹ and R² are each independently a) hydrogen or b) (C₁-C₄)alkyl;
X⁵ is —CH₃ or —CF₃.
in the manufacture of a medicament for the palliative, prophylactic or curative treatment of negative energy balance in ruminants.

2. Use of a compound of claim 1 wherein
X¹ and X² are each independently a) hydrogen, b) —CF₃, c) —OCF₃, d) (C₁-C₄)alkyl, e) —OCH₃ or f) halo;
X³ is —Y—C(R¹)(R²)—COOH and X⁴ is hydrogen;
Y is —O—;
X¹ is —CH₃.

3. Use of a compound selected from the group consisting of:
2-Methyl-2-{2-[4-methyl-2-(4-trifluoromethyl-phenyl)-thiazole-5-carbonyl]-1,2,3,4-tetrahydro-isoquinolin-6-yloxy}-propionic acid;
2-{2-[2-(4-Methoxy-phenyl)-4-methyl-thiazole-5-carbonyl]-1,2,3,4-tetrahydro-isoquinolin-6-yloxy}-2-methyl-propionic acid;
2-Methyl-2-{2-[4-methyl-2-(3-trifluoromethyl-phenyl)-thiazole-5-carbonyl]-1,2,3,4-tetrahydro-isoquinolin-6-yloxy}-propionic acid; and
52-Methyl-2-{2-[4-methyl-2-(2-trifluoromethyl-phenyl)-thiazole-5-carbonyl]-1,2,3,4-tetrahydro-isoquinolin-6-yloxy}-propionic acid;
or a prodrug of said compound or a pharmaceutically acceptable salt of said compound or prodrug.

4. The use according to any one of claims 1 to 3 for the palliative, prophylactic or curative treatment of ruminant disease associated with negative energy balance in ruminants.

5. The use according to claim 4 wherein the ruminant disease associated with negative energy balance in ruminants is selected from fatty liver syndrome, dystocia, immune dysfunction, impaired immune function, toxification, primary ketosis, secondary ketosis, downer cow syndrome, indigestion, inappetence, retained placenta, displaced abomasum, mastitis, (endo-)-metritis, infertility, low fertility, and lameness.

6. The use according to any one of claims 1 to 5 wherein the compound of formula I is administered during the period from 30 days prepartum to 70 days postpartum.

7. The use according to claim 5 wherein the compound of formula I is administered up to three times during the first seven days postpartum.

8. The use according to claim 7 wherein the compound of formula I is administered once during the first 24 hours postpartum.

9. The use as claimed in any one of claims 1 to 8 in the manufacture of a medicament to increase ruminant milk quality and/or milk yield.

10. The use according to claim 9 wherein the ruminant is a dairy cow.

11. The compound of formula 1 as described in any one of claims 1 to 3, for use in the palliative, prophylactic or curative treatment of negative energy balance in ruminants.

12. The compound of formula 1 as described in any one of claims 1 to 3, for use in the palliative, prophylactic or curative treatment of ruminant disease associated with negative energy balance in ruminants.

13. The compound of formula I, for use in the palliative, prophylactic or curative treatment of negative energy balance in ruminants, and for increasing ruminant milk quantity and/or quality.
